(19) United States
(12) Patent Application Publication
(10) Pub. No.: US 2011/0197900 A1
(43) Pub. Date: Aug. 18, 2011

(54) PERCUTANEOUS INTERVENTIONAL CARDIOLOGY SYSTEM FOR TREATING VALVULAR DISEASE

(76) Inventor: Richard R. Heuser, Phoenix, AZ (US)

(21) Appl. No.: 13/092,041

(22) Filed: Apr. 21, 2011

Related U.S. Application Data
(62) Division of application No. 11/688,785, filed on Mar. 20, 2007.

(51) Int. Cl. A61B 17/00

(52) U.S. Cl. 128/898

(57) ABSTRACT

A system is provided for treating valvular disease percutaneously though a ventricular apex of the heart. The system includes a needle for piercing through the ventricular apex of the heart and creating a hole whereby a catheter can be passed through the skin and the wall of the heart to gain access to the interior of the heart. Once percutaneous access to the heart is obtained, any of a variety of techniques can be utilized for annular and/or valvular therapy and/or repair through, the catheter. A closure device is necessary for closing the puncture of the heart. Any of a variety of vascular closure devices can be modified to be utilized with the system to close the puncture of the ventricular apex of the heart.

### BACKGROUND

This invention relates to a system for treating valvular disease of the heart percutaneously through a ventricular apex of a heart.

Percutaneous valve therapies are minimally invasive techniques for treating patients with heart valve problems. The traditional catheter based approach involves accessing the heart valve through the femoral vein and/or artery in the groin. Worldwide, three to four million of such catheter procedures are currently being done yearly. As these procedures are performed on older patients with more complicated health issues including peripheral vascular disease, complications from vascular access have become more common. Several vascular closure devices and anticoagulation methods have been developed to more adequately and quickly seal a percutaneous approach in the femoral vein and/or artery to combat the problems encountered with such vascular access to the heart. Alternatively, many of the problems encountered with vascular access to the heart could be avoided with direct access to the heart. However, open heart surgery is not considered minimally invasive and can lead to far greater complications than encountered with traditional catheter based approaches through the groin. An interventional percutaneous approach for treating valvular disease of the heart by directly accessing the heart would avoid the complications of vascular catheter access to the heart, while still gaining the advantages of a minimally invasive valve treatment over open heart surgery.

### SUMMARY OF THE INVENTION

The invented system and method provides for treating valvular disease percutaneously though a ventricular apex of the heart. The system includes a needle for piercing through the ventricular apex of the heart and creating a hole whereby a catheter can be passed through the skin and the wall of the heart to gain access to the interior of the heart. Other piercing tools could be utilized including any of a variety of needle types. Alternatively, a catheter with a distal end having a piercing edge could be utilized in place of the needle and catheter combination. Once percutaneous access to the heart is obtained, any of a variety of techniques can be utilized for annular and valvular therapy and/or repair. A closure device is necessary for closing the puncture of the heart. Any of a variety of vascular closure devices can be modified to be utilized to close the puncture of the ventricular apex of the heart and are described below. The myocardium wall of the apex of the heart is not as thick as other areas and therefore offers a preferred approach for direct access into the heart. However, other locations of the heart wall could be used for percutaneous access.

Further, while commonly referred to as percutaneous access, the invention described herein could also be referred to as minimally invasive as there is a fair amount of distance in the subxiphoid territory to the apex of the heart. Therefore a mini thoracotomy could be done in a subxiphoid territory with surgical access to the apex of the heart, at which point direct puncture into the heart with the needle would be possible. Such a minimally invasive procedure would still be preferable to a fully invasive procedure and/or a vascular approach. An open ventricular incision or percutaneous approach to the heart could allow for better closure than a surgical approach, offer a less invasive procedure, limit or prevent oozing, and be an easily repeatable procedure with minimal side effects.

### BRIEF DESCRIPTION OF THE DRAWINGS

FIG. 1A is a partial cross-sectional view a heart showing a needle entering the left ventricle through the left ventricular apex wall.

FIG. 1B is a partial cross-sectional view of a heart showing a catheter entering the left ventricle through the hole in the left ventricular apex wall created by the needle shown in FIG. 1A.

FIG. 2A is a partial cross-sectional view of a heart showing a needle entering the left ventricle through the left ventricular apex wall and proceeding through the mitral valve to the left atrium.

FIG. 2B is a partial cross-sectional view of a heart showing a catheter entering the left ventricle through the hole in the left ventricular apex wall created by the needle shown in FIG. 2A.

FIG. 3A is a partial cross-sectional view of a heart showing a valve repair system inserted through the catheter shown in FIG. 2B and into the left ventricle, mitral valve, and left atrium of the heart.

FIG. 3B is a close-up partial cross-sectional view of the mitral valve area of the heart showing the valve repair system depicted in FIG. 3A.

FIG. 4A is a partial cross-sectional view of a heart showing another valve repair system inserted through the catheter shown in FIG. 2B.

FIG. 4B is a close-up partial cross-sectional view of the mitral valve area of the heart showing the valve repair system depicted in FIG. 4A.

FIG. 5A is a partial cross-sectional view of a heart showing yet another valve repair system inserted through the catheter shown in FIG. 2B.

FIG. 5B is a close-up partial cross-sectional view of the mitral valve area of the heart showing the valve repair system depicted in FIG. 5A.

FIG. 6A is a partial cross-sectional view of a heart showing a vascular closure device modified to be operable to close the hole in the ventricular apex wall of the heart created by the needle and catheter depicted in FIGS. 1-5.

FIG. 6B is a close-up partial cross-sectional view of the ventricular apex wall area of the heart showing the closure device depicted in FIG. 6A.

FIG. 7A is a partial cross-sectional view of a heart showing another vascular closure device modified to be operable to close the hole in the ventricular apex wall of the heart created by the needle and catheter depicted in FIGS. 1-5.

FIG. 7B is a close-up partial cross-sectional view of the ventricular apex wall area of the heart showing the closure device depicted in FIG. 7A.

FIG. 8A is a partial cross-sectional view of a heart showing yet another vascular closure device modified to be operable to close the hole in the ventricular apex wall of the heart created by the needle and catheter depicted in FIGS. 1-5.

FIG. 8B is a close-up partial cross-sectional view of the ventricular apex wall area of the heart showing the closure device depicted in FIG. 8A.

FIG. 9A is a partial cross-sectional view of a heart showing still yet another vascular closure device modified to be operable to close the hole in the ventricular apex wall of the heart created by the needle and catheter depicted in FIGS. 1-5.

FIG. 9B is a close-up partial cross-sectional view of the ventricular apex wall area of the heart showing the closure device depicted in FIG. 9A.

FIG. 10 is a cross-sectional view of a heart showing a needle entering the right ventricle through the right ventricular apex wall.

### DETAILED DESCRIPTION OF THE PREFERRED EMBODIMENTS

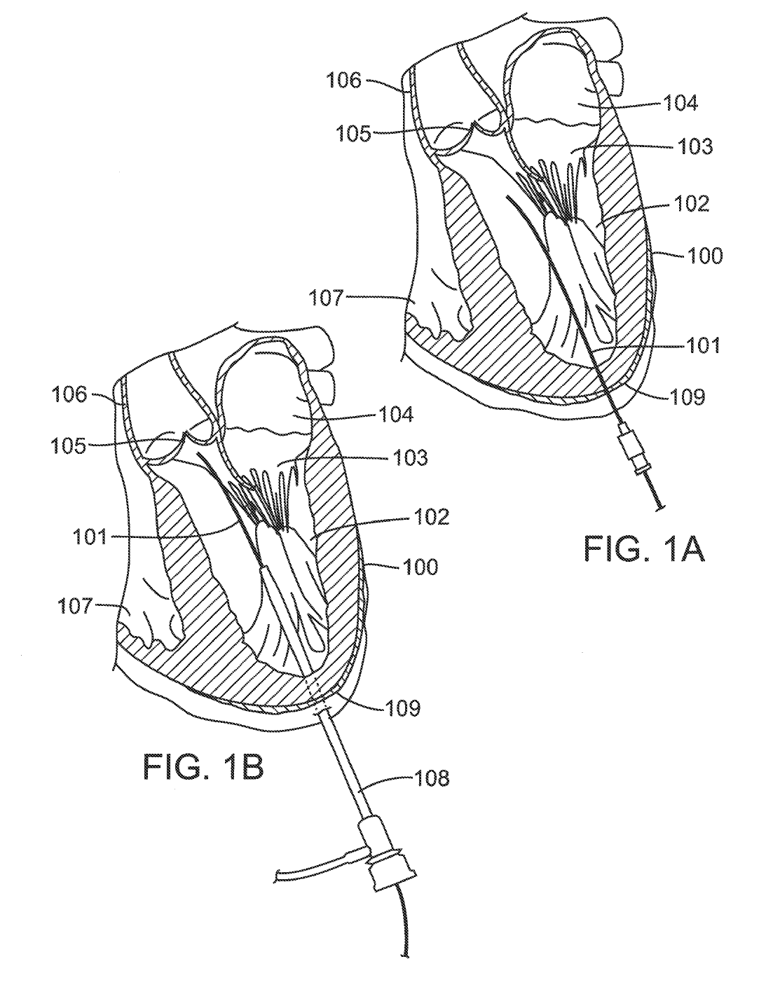

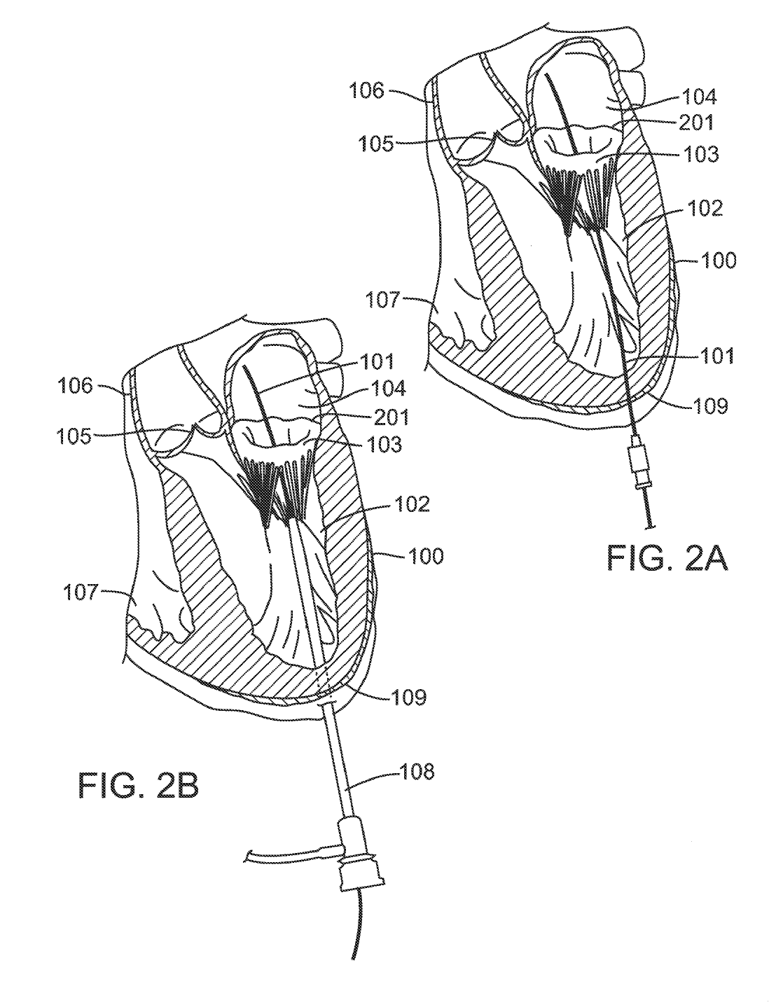

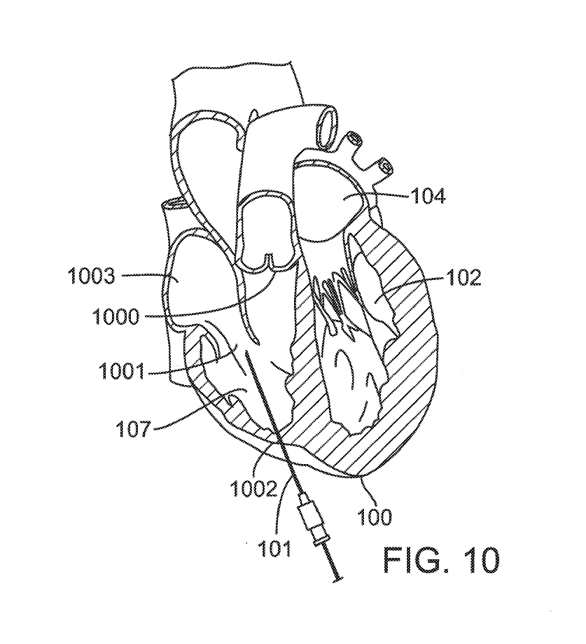

As shown in FIG. 1A, a needle 101 can be inserted into the left ventricle 102 of the heart 100 via the left ventricular apex wall of the heart 109. Needle 101 can be inserted percutaneously or minimally invasively via a mini thoracotomy to allow direct needle insertion access to the heart. Also illustrated for context in FIG. 1 are left atrium 104, mitral valve 103, aorta 106, aortic valve 105 and right ventricle 107. As shown in FIG. 1B, once needle 101 has been inserted into left ventricle 102, a catheter 108 can be inserted through the hole created by the needle 101. For example, a small (e.g. 4 or 6-French catheter) can be inserted and then a time period can pass until hemostasis is obtained so that there is no bleeding around the outside of the heart. Then, similar to a femoral artery approach, one can dilate to a larger size catheter (e.g. up to 36-French for valvular therapy, such as therapy for the aortic valve). As shown in FIG. 2A, needle 101 can be inserted into the left ventricle 102 of the heart 100 via the left ventricular apex wall of the heart 109 and positioned through a valve of a heart (e.g., mitral valve 103) and into an atrium of the heart (e.g. the left atrium 104) to allow for treatment of valvular disease. As shown in FIG. 2B, once needle 101 has been inserted into left ventricle 102, a catheter 108 can be inserted through the hole created by the needle 101. Dilation, as described above, can occur allowing for a catheter of a size suitable for allowing a valve repair system access into the heart. Alternatively, needle 101 could be positioned through aortic valve 105 and into aorta 106. Similarly, as shown in FIG. 10 and described below, needle 101 can access right ventricle 107 of the heart 100 in order to treat valvular disease of the tricuspid valve and/or pulmonic valve.

A variety of valve repair systems can be utilized once catheter access to the heart is obtained. These include any number of devices compatible to be inserted through a catheter and operable within the heart, including sheaths operable to open and close when embedded within a valve of the heart, clips (e.g. an E-Valve device or Edwards Clip Device) that are operable to open and close when embedded within a valve of the heart, radiofrequency devices (e.g., the Boa System™, Boa-Surg Device™, and Boa-Cathe Device™ manufactured by QuantumCor, Inc. of San Clemente, Calif. and hereto referred to collectively as the “QuantumCor” device) operable to apply radiofrequency energy to an annulus of a valve of the heart (e.g., annulus 201 of mitral valve 103), suturing devices operable to suture one or more leaflets of a valve of the heart, cinching devices operable to cinch an annulus of a valve of the heart, and replacement valves operable to replace a valve of the heart. Similarly, any open surgical technique or vascular percutaneous technique (e.g., an Alfieri technique, Inoue technique for mitral valvuloplasty, or any other annular or valvular therapy such as standard valvuloplasty of the aortic valve) can be utilized through a catheter placed directly into the heart as described above.

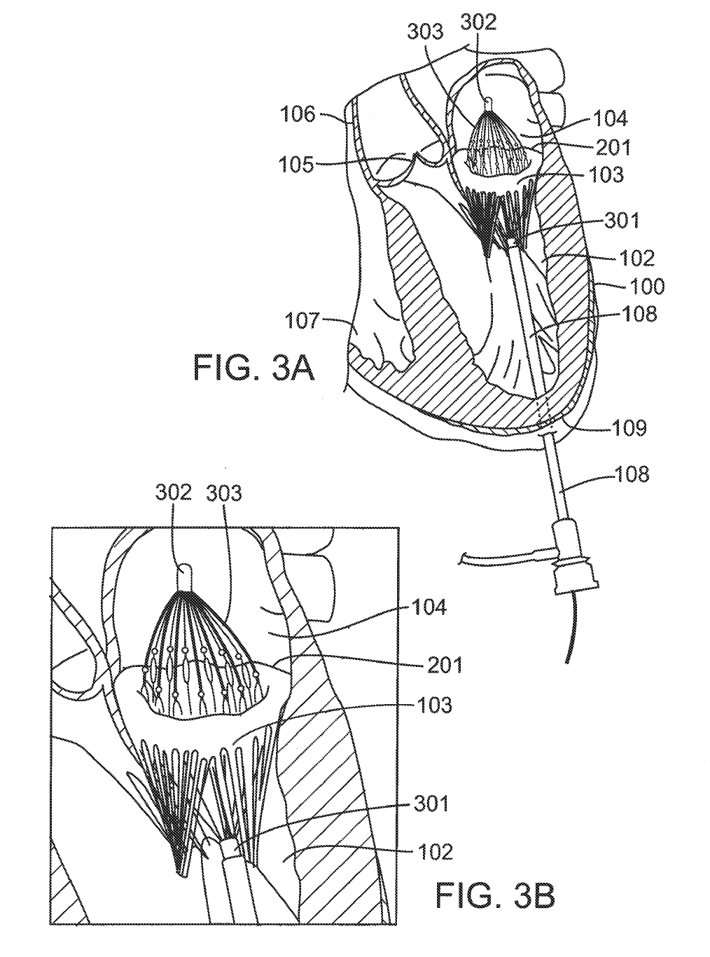

A sheath (e.g., a modified QuantumCor device) compatible to be inserted through a catheter and inserted into a ventricle of a heart is shown in FIGS. 3A and 3B. The sheath could be flexible to allow the valve to open and close the thermisters, transducers, or electrodes of the sheath. Such thermisters, transducers or electrodes in the upper mid portion of the wire basket of the sheath could be utilized to apply radiofrequency energy to the annular region of the valve (e.g., the mitral annular region). Application of radiofrequency energy to the mitral annulus (a collagen-rich territory of the mitral valve that supplies the structure and framework for support of the mitral valve) can cause shrinking of the collagen, thereby treating mitral regurgitation. As shown in FIG. 3A, a catheter 108 has been inserted through a hole created by needle 101 as shown in FIG. 2B and described above. Sheath 303 can be inserted into catheter 108 in a closed position to allow it to fit through the catheter and be placed into a position in the heart within mitral valve 103 with sheath distal end 302 in left atrium 104 and sheath proximal end 301 in left ventrical 102. The sheath distal end 302 can be pulled closer to sheath proximal end 301 utilizing a guide wire (not shown) within sheath 303 or any other means, thereby causing the expansion of sheath 303 to catch and/or fit within mitral valve 103.

Alternatively, other means for expanding sheath 303 and/or inserting sheath 303 into position within a valve can be utilized. For example, in an alternative embodiment illustrated using the same FIGS. 3A and 3B, a hollow tube 301 with a cap 302 could be utilized, whereby sheath 303 is compacted to fit within hollow tube 301 and passed through hollow tube 301 until it hits cap 302. Sheath 303 can be pushed through cap 302, in the process attaching cap 302 to sheath 303, and moved into position. Sheath 303 would expand as it exits tube 301 with cap 302 attached to sheath 300 at the tip. Once sheath 303 is in position, tube 301 can be removed. Alternative systems and techniques for embedding sheaths into a valve of the heart through a catheter can be used, including any such systems and techniques commonly used during vascular access to treating valvular disease of the heart.

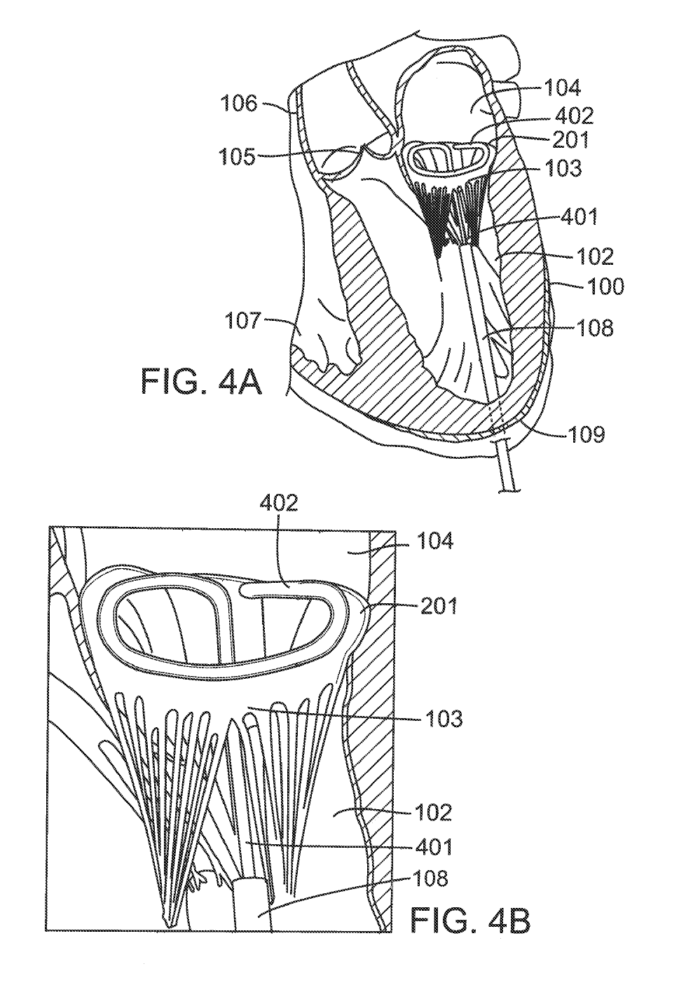

Other sheath and radiofrequency devices can be inserted into catheter 108 into a valve of the heart to treat valvular disease. For example, as shown in FIGS. 4A and 4B, another variant of the QuantumCor device is shown. In this embodiment, radiofrequency device 401 with coils 402 spread at the site of the annulus 201 is positioned into place in mitral valve 103. This can be done utilizing techniques as described above in the placement of sheath 303 or via maneuvering radiofrequency device 401 and coils 402 into position in the heart. A sheath can also be placed in combination with radiofrequency device 401.

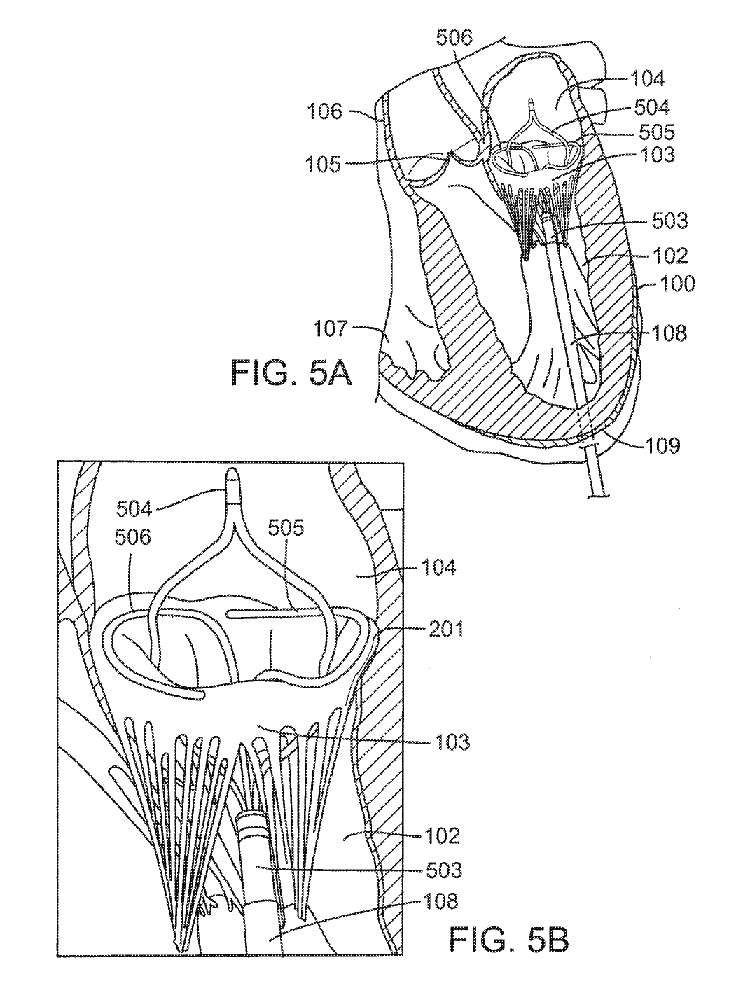

FIGS. 5A and 5B illustrate another variant of the QuantumCor device. Radiofrequency device 503 can include coils 505 and 506 placed at the site of the annulus 201 of a valve (e.g., the mitral valve 103 as shown) and a mid position flexible notched structure 504 configured to catch on the mitral valve. Coils 505 and 506 and/or notched structure 504 can be radiofrequency transducers or electrodes for application of radiofrequency energy to the valve (e.g., mitral valve 103 as shown). This can be done utilizing techniques as described above in the placement of sheath 303 or via maneuvering radiofrequency device 503 including coils 505 and 506 and notched structure 504 into position in the heart. A sheath can also be placed in combination with radiofrequency device 503.

Any number of other valvular treatments can be utilized via access to the valves of the heart through direct or percutaneous needle and catheter puncture through a ventricular apex wall. Treatments include placing clips with valves, utilizing suturing and or cinching devices for surgical methods of treatment, and/or replacing existing damaged or developmentally faulty valves with suitable replacement valves (e.g., porcine or other animal valves, bioengineered valves, donated human valves, and tissue engineered valves). Sheaths, radiofrequency, and other devices inserted into valves and operable to open and close within the valve can be made with Nitinol or other flexible metal materials. Any such treatments can be visually aided through the use of monitoring systems including intracardiac echocardiography (ICE), transesophageal echocardiography (TEE), fluoroscopy (“fluoro”) or any other monitoring means.

After applying treatment to one or more valves, the puncture of the ventricular apex wall must be closed. Any variety of vascular closure systems utilized in femoral vein and/or artery or other vascular access to the heart methods can be modified for use in closing up the puncture of ventricular apex wall of the heart. Modifications to existing vascular closure devices can include enlargement of existing devices to allow for closing a hole made by up to a 36-French catheter, the addition of thrombin and/or collagen to result in more adequate hemostasis, and/or the materials which closure devices are currently made of replaced with materials which are reabsorbable over time by the body.

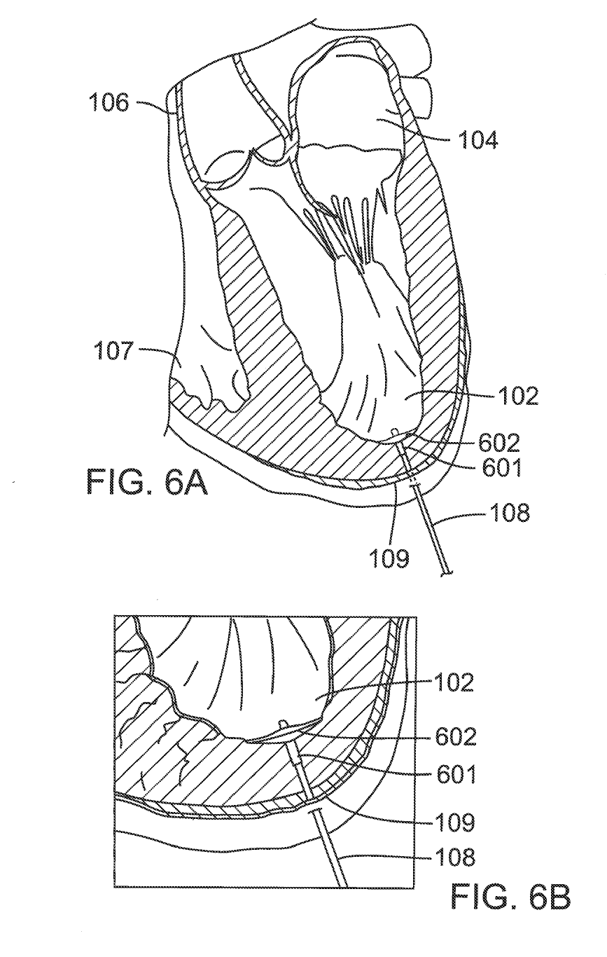

For example, a Boomerang™ wire vascular closure device (manufactured by Cardiva Medical, Inc. of Mountain View, Calif.) can be utilized in the disclosed system for treating valvular disease percutaneously as shown in FIGS. 6A and 6B. Wire 601 with disc 602 attached on the end can be inserted through catheter 108 into the ventricle of the heart (e.g. the left ventricle 102 as shown) and with tension put into place to block the hole created by the needle and catheter. After a period of time (e.g., fifteen to thirty minutes), hemostasis would be achieved. Disc 602 would have to be modified to be larger than the vascular disc device currently on the market, perhaps instead of 016, as large as 035, 038 or even up to 064.

Alternatively, a Perclose® vascular closure device (manufactured by Abbott Vascular, a division of Abbott, of Redwood City, Calif.) can be utilized in the disclosed system for treating valvular disease percutaneously. Such a device could be modified to be more efficiently sized to treat a percutaneous puncture of the heart. Similary, a Chito-Seal™ topical hemostasis pad (manufactured by Abbott Vascular, a division of Abbott, of Redwood City, Calif.) can be utilized as a closure device in the disclosed system for treating valvular disease percutaneously. Other pads and or patches could also be modified and utilized as a closure device to improve thrombosis or hemostasis in the disclosed system for treating valvular disease percutaneously, including, for example, the Syvekpatch® (manufactured by Marine Polymer Technologies, Inc. of Danvers, Mass.).

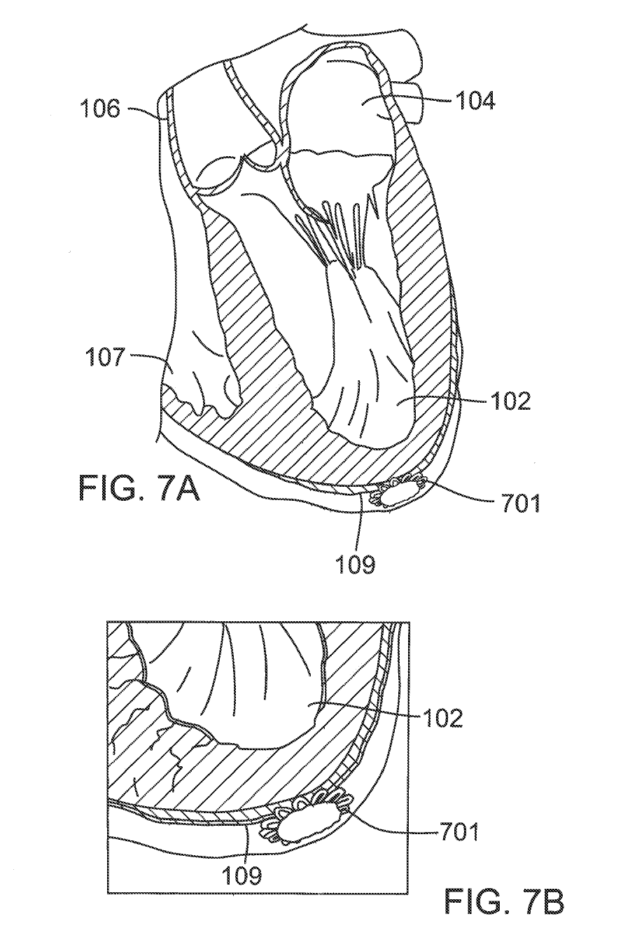

Another vascular closure system that can be modified and utilized in the disclosed system for treating valvular disease percutaneously is the Starclose® vascular closure device (manufactured by Abbott Vascular, a division of Abbott, of Redwood City, Calif.) as shown in FIGS. 7A and 7B. The Starclose® closure device 701 shown is an enlarged version of the vascular version currently on the market. Such enlargement would be necessary to allow for closure of a hole in the heart left by an up to 36-French catheter. One advantage of a modified Starclose® device is that while at first the closure device is inside the endocardial surface of the heart, it is pulled through the wall of the heart so the closing seal would occur outside the heart. The advantage of the puncture seal being outside of the heart is that nothing is left for blood to possibly contact inside the heart muscle, resulting in lower likelihood of thrombus formation or embolization of a clot in the heart leading to a stroke. Modifications to the Starclose® device for utilization in the disclosed system include enlargement of the Nitinol clip and possible replacement of Nitinol with reabsorbable material such as suture type material or other material that dissolves over a period of weeks or months.

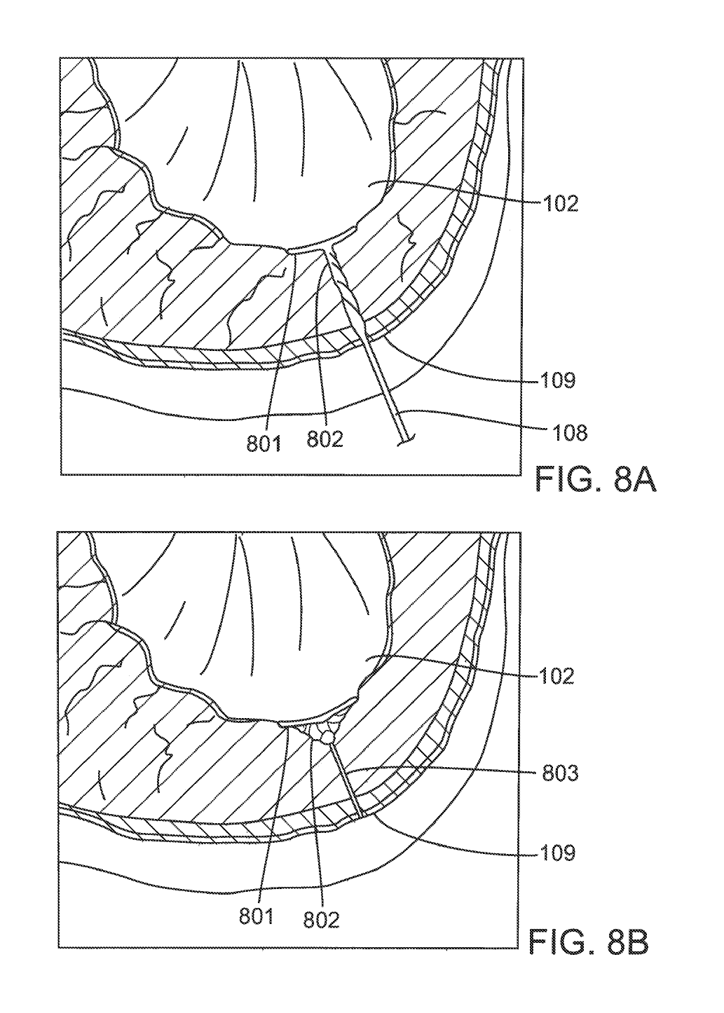

Yet, another vascular closure system that can be modified and utilized in the disclosed system for treating valvular disease percutaneously is the Angio-seal® vascular closure device (manufactured by St. Jude Medical, Inc., of St. Paul, Minn.) as shown in FIGS. 8A and 8B. Anchor 801 of Angio-seal® can be inserted through catheter 108 into the left ventricle 102 along with collagen sponge 802 forming a seal of the puncture hole 803 left behind when catheter 108 is removed. The Angio-seal® device would need to be modified to be used up to a 36-French size hole instead of the 6-French or 8-French size for sealing blood vessel punctures. Modifications include increasing the size of anchor 801 and the size of collagen sponge 802, and perhaps including thrombin to the device. Further, the Angio-seal® device could be combined with other therapies and closure devices when utilized in the disclosed system.

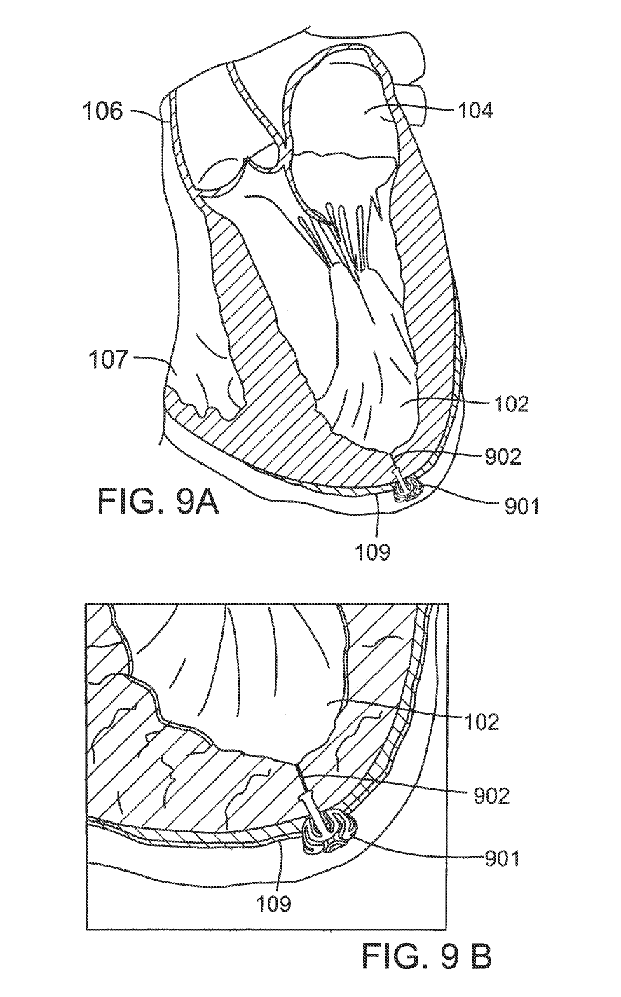

Still yet another vascular closure system that can be modified and utilized in the disclosed system for treating valvular disease percutaneously is the AngioLink® vascular closure system (manufactured by Medtronic, Inc., of Minneapolis, Minn.) as shown in FIGS. 9A and 9B. To accommodate some of the valvular procedures described, AngioLink® would need to be modified to accommodate a hole left by up to a 36-French catheter. As shown in FIGS. 9A and 9B, AngioLink® staple 901 is attached to ventricular apex wall 109, sealing hole 902 left by removal of catheter 108.

While FIGS. 1-9 illustrate the disclosed system for treating valvular disease percutaneously through the left ventricular apex of the heart, the system can also be utilized for valvular repair through the right ventricular apex. As shown in FIG. 10, needle 101 can puncture right ventricular apex wall 1002 of heart 100 and enter right ventricle 107. Similar to the procedures described above, a catheter (not shown) can be inserted through the hole created by needle 101 to allow for a valve repair system (not shown, but any of the previously described systems can be used) to be inserted into the heart and utilized. Access into the right ventricle 107 allows for access into the right atrium 1003 and repair of the pulmonic valve 1000 and/or the tricuspid valve 1001.

It is believed that the disclosure set forth above encompasses multiple distinct inventions with independent utility. While each of these inventions has been disclosed in its preferred form, the specific embodiments thereof as disclosed and illustrated herein are not to be considered in a limiting sense as numerous variations are possible. The subject matter of the inventions includes all novel and non-obvious combinations and subcombinations of the various elements, features, functions and/or properties disclosed herein. No single feature, function, element or property of the disclosed embodiments is essential to all of the disclosed inventions. Similarly, where the claims recite “a” or “a first” element or the equivalent thereof, such claims should be understood to include incorporation of one or more such elements, neither requiring nor excluding two or more such elements.

It is believed that the following claims particularly point out certain combinations and subcombinations that are directed to one of the disclosed inventions and are novel and non-obvious. Inventions embodied in other combinations and subcombinations of features, functions, elements and/or properties may be claimed through amendment of the present claims or presentation of new claims in this or a related application. Such amended or new claims, whether they are directed to a different invention or directed to the same invention, whether different, broader, narrower or equal in scope to the original claims, are also included within the subject matter of the inventions of the present disclosure.

1-30. (canceled)

31. A method for treating valvular disease, the method comprising:
inserting a needle through the skin and a ventricular apex of the heart;
inserting a catheter into a ventricle of the heart through a hole in the ventricular apex of the heart created by inserting the needle;
inserting a valve repair device through the catheter and into the ventricle of the heart;
utilizing the valve repair device within the heart to repair a diseased valve;
inserting a closure device through the catheter; and
closing the hole in the ventricle of the heart created by the needle and catheter with the closure device.

32. The method of claim 31, wherein inserting a catheter into a ventricle of the heart through a hole in the ventricular apex of the heart created by inserting the needle comprises inserting a catheter suitable for insertion into an atrium of the heart, a pulmonary artery, or an aorta.

33. The method of claim 31, wherein inserting a valve repair device through the catheter comprises inserting at least one of:
a sheath compatible to be inserted through the catheter and operable to open and close when embedded within a valve of the heart;
a clip compatible to be inserted through the catheter and operable to open and close when embedded within a valve of the heart;
a radiofrequency device compatible to be inserted through the catheter and operable to apply radiofrequency energy to an annulus of a valve of the heart;
a suturing device compatible to be inserted through the catheter and operable to suture one or more leaflets of a valve of the heart;
a cinching device compatible to be inserted through the catheter and operable to cinch an annulus of a valve of the heart; and
a replacement valve compatible to be inserted through the catheter and operable to replace a valve of the heart.

34. The method of claim 31, wherein inserting a needle through a ventricular apex of the heart comprises inserting a needle through the left ventricular apex.

35. The method of claim 34, wherein the valvular disease is aortic valve disease or mitral valve disease.

36. The method of claim 31, wherein utilizing the valve repair device within the heart to repair a diseased valve comprises utilizing the valve repair device within the heart to repair an aortic valve or a mitral valve.

37. The method of claim 31, wherein inserting a needle through a ventricular apex of the heart comprises inserting a needle through a right ventricular apex.

38. The method of claim 37, wherein the valvular disease is tricuspid valve disease or pulmonic valve disease.

39. The method of claim 31, wherein utilizing the valve repair device within the heart to repair a diseased valve comprises utilizing the valve repair device within the heart to repair a tricuspid valve or a pulmonic valve.

40. The method of claim 31, wherein insertine a sheath compatible to be inserted through the catheter and operable to open and close when embedded within a valve of the heart comprises inserting a sheath configured to be embedded within the valve via one or more notches configured to catch on an annulus of a valve.

41. The method of claim 31, wherein inserting a catheter into a ventricle of the heart through a hole in the ventricular apex of the heart created by inserting the needle comprises inserting a catheter of at least 4-French size and not more than 36-French size.

42. The method of claim 31, wherein inserting a closure device through the catheter comprises inserting a vascular closure device modified to close a diameter of the hole created by the needle and/or catheter.

43. The method of claim 31, wherein inserting a closure device through the catheter comprises inserting a closure device that is coated with thrombin and/or collagen to aid in hemostasis.

44. The method of claim 31, wherein inserting a closure device through the catheter comprises inserting a closure device that is made of material that is reabsorbable by the body after hemostasis occurs.

45. The method of claim 31, further comprising using a monitoring system to monitor a heart function parameter.

46. The method of claim 45, wherein using a monitoring system to monitor a heart function parameter comprises performing intracardiac echocardiography, transesophageal echocardiography, or fluoroscopy.
